(19) United States
(12) Patent Application Publication
(10) Pub. No.: US 2005/0054723 A1
(43) Pub. Date: Mar. 10, 2005

(54) Method for the treatment of glaucoma and ocular hypertension with prostaglandin analogues without melanogenic side effect

(76) Inventor: Johan Stjernschantz, Uppsala (SE)

Correspondence Address: DINSMORE & SHOHL, LLP, 1900 CHEMED CENTER, 255 EAST FIFTH STREET, CINCINNATI, OH, 45202, US

(21) Appl. No.: 10/847,254

(22) Filed: May 17, 2004

Related U.S. Application Data
(60) Provisional application No. 60/473,191, filed on May 27, 2003.

(51) Int. Cl. A61K 31/557

(52) U.S. Cl. 514/530; 514/573

(57) ABSTRACT

Prostaglandin-induced increased pigmentation of the iris can be avoided, or significantly reduced, when using a selective EP₂ prostanoid receptor agonist in combination with an alpha-adrenergic agonist. Methods and compositions for the treatment of glaucoma and ocular hypertension are described.

### RELATED APPLICATION

The present application claims priority under 35 U.S.C. § 119 of U.S. application Ser. No. 60/473,191 filed May 27, 2003.

### FIELD OF THE INVENTION

The present invention relates to a method whereby increased iridial pigmentation, occurring as a side effect in topical prostaglandin treatment can be avoided, or at least largely reduced. The invention also concerns ophthalmic compositions for this purpose.

### BACKGROUND

Glaucoma is an eye disorder usually associated with elevated intraocular pressure (IOP). The elevated IOP is believed to be detrimental to the optic nerve and the retina, causing an excavation of the optic nerve head and defects in the visual field. Several drugs are clinically used to treat glaucoma, e.g. cholinergic drugs, carbonic anhydrase inhibitors, beta-adrenergic antagonists, and prostaglandins. All these drugs work by reducing the elevated pressure in the eye.

Prostaglandin analogues are widely used for the treatment of glaucoma, and all analogues currently on the market cause increased pigmentation of the iris as a side effect in predisposed patients. Currently there are four prostaglandin analogues in clinical use for glaucoma treatment, namely latanoprost (Xalatan^{R}; Pfizer, USA), travoprost (Travatan^{R}; Alcon, USA), bimatoprost (Lumigan^{R}; Allergan, USA) and isopropyl unoprostone (Rescula^{R}; Ueno Fine Chemicals, Japan). All these drugs are analogues of PGF_{2α} and they are likely to work mainly by stimulating the FP prostanoid receptor. Information about the drugs can be found e.g. in the articles by Stjernschantz, 2001; Hellberg et al., 2001; Woodward et al, 2001, and Yamamoto et al., 1997.

The prostaglandins are fatty acids usually derived from the precursor eicosatetraenoic acid or arachidonic acid through an enzymatic process catalyzed by the cyclo-oxygenase enzyme. Typically the prostaglandins carry a cyclopentane ring to which two side chains attach, the upper side being the alpha chain comprising a 7 carbon carboxy-terminated aliphatic chain, whereas the lower side chain, the omega chain conists of 8 carbons with a terminal methyl group. The prostaglandins are classified into different subgroups: A, B, C, D, E, F, G, H, I and J depending on the structure and the substituents in the cyclopentane ring. Subscripts 1-3 are used depending on the number of double bonds in the side chains. A review of the prostaglandin chemistry and pharmacology can be found e.g. in “Goodman and Gilman's The Pharmacological Basis of Therapeutics”, Goodman et al. (Eds.), McGraw-Hill Professional, 9^{th} Ed., 1996.

Bimatoprost (17-phenyl-18,19,20-trinor PGF_{2α} ethylamide) has also been claimed to act as a prostamide, but current evidence suggest that the compound may as well work as a prostaglandin with potent action on the FP prostanoid receptor (Hellberg et al., 2003).

The prostaglandins act on specific prostanoid receptors of the 7 TM (transmembrane) G-protein coupled metabotropic type. The receptors are subdivided into 8 classes; DP (PGD₂), EP₁ (PGE₂), EP₂ (PGE₂), EP₃ (PGE₂), EP₄ (PGE₂), FP (PGF_{2α}), IP (PGI₂) and TP (TxA₂), the naturally occurring ligand of each receptor being indicated in parenthesis after each receptor. It should be emphasized that many of the naturally occurring prostaglandins are rather unselective and stimulate many of the above-mentioned receptors. Importantly, these receptors mediate distinct effects, and thus by using selective receptor agonist the vast number of biologic effects that the naturally occurring prostaglandins exert, can be reduced to one, or just a few. Thus most of the prostaglandin analogues in clinical use for glaucoma treatment are selective FP receptor agonists because the FP prostanoid receptor is not involved in the nociceptive, irritative effect, commonly seen by naturally occurring prostaglandins in the eye (Stjernschantz, 2001).

As mentioned above an annoying side effect of the prostaglandins in clinical use is that they cause increased pigmentation of the iris in susceptible individuals (Stjernschantz et al., 2002). This effect is based on the ability of the prostaglandins to stimulate iridial melanocytes to produce pigment (Stjernschantz et al., 2002). Although the side effect does not appear to lead to any harmful consequences it nevertheless constitutes a significant problem since the underlying mechanism is not completely understood. The color change of the eye also seems to be irreversible, or only very slowly reversible (Stjernschantz et al., 2002). Thus, it would be a clear advantage if prostaglandin analogues without the above mentioned melanogenic side effect could be developed for clinical use. Patent applications dealing with new inventions concerning how to avoid the prostaglandin-induced iris pigmentation have previously been filed, see for example PCT/SE99/01993 (Method for preventing increased iridial pigmentation during prostaglandin treatment, J. Stjernschantz and B. Resul, published on May 11, 2000 as WO 00/25771).

### SUMMARY OF THE INVENTION

The present inventor has unexpectedly found a possible solution to the above mentioned problem of prostaglandin-induced increased iris pigmentation, the solution being that prostaglandins of E type with selectivity for the EP₂ prostanoid receptor should be used for IOP reduction, and that such prostaglandin analogues should be combined with alpha-adrenergic agonists as explained in more detail below. The invention makes available methods and compositions as defined in the attached claims, hereby incorporated by reference.

### DETAILED DESCRIPTION OF THE INVENTION

For convenience, certain terms employed in the specification, examples, and appended claims are collected here.

The phrase “pharmaceutically acceptable” refers to molecular entities and compositions that are physiologically tolerable and do not typically produce an allergic or similar unwanted reaction, such as pain, irritation etc, when administered to a patient.

The phrase “therapeutically effective amount” is used herein to mean an amount sufficient to suppress to some beneficial degree, preferably to reduce by at least about 30 percent, more preferably by at least 50 percent, most preferably by at least 90 percent, the disease in question, e.g. glaucoma or ocular hypertension.

“Treatment” shall mean preventing or lessening glaucoma or ocular hypertension, including the lessening in severity of said diseases. Any amelioration of any symptom of glaucoma or ocular hypertension pursuant to treatment using a method or composition according to the invention falls within the scope of the invention.

The term “prodrug” is used in the widest sense of the word, meaning any precursor of a therapeutically active substance. A prodrug must undergo chemical conversion by metabolic processes before becoming an active pharmacological agent.

The terms “receptor” and “agonist” are to be understood as generally accepted by a person skilled in the art. The prostanoid receptors, respectively, are well defined, cloned, sequenced, and pharmacologically characterized entities. Agonists on the receptors accordingly are compounds (prostaglandin analogues) that bind and activate the receptors. Selective agonists on the receptors are compounds (prostaglandin analogues) that with preference bind and activate the receptors over other prostanoid receptors, in pharmacological terms usually meaning that the difference in EC₅₀ or KD value between the receptor in question, and other prostanoid receptors being at least one log unit.

The naturally occurring prostaglandins such as e.g. PGF_{2α} and its isopropyl ester prodrug cause the following side-effects when applied topically on the eye: irritation, comprising e.g. smarting, foreign body sensation and lacrimation, conjunctival hyperemia and increased pigmentation of the iris (Stjernschantz, 2001). The present inventor has previously found out that the FP and the EP₂ prostanoid receptors are not involved in the nociceptive response to the naturally occurring prostaglandins whereas virtually all the other prostanoid receptors mediate pain (Stjernschantz, 2001).

Surprisingly, in the work conducted under the supervision of the present inventor, it was found that whereas the FP prostanoid receptor is consistently expressed by human iridial melanocytes, i.e. the cells that produce the pigment causing the iris to appear darker, the EP₂ prostanoid receptor is not expressed in the cells (Wentzel et al., 2003). Thus, analogues that stimulate selectively the EP₂ prostanoid receptor are very unlikely to cause increased pigmentation of the iris simply because the target cells for the side-effect do not express the necessary receptor. On the contrary, selective FP receptor agonists such as latanoprost, travoprost and bimatoprost are very likely to cause increased pigmentation of the iris in susceptible individuals because the FP prostanoid receptor is expressed by the iridial melanocytes. Since the EP₂ prostanoid receptor does not mediate pain, analogues selective for the EP₂ receptor will cause neither increased pigmentation of the iris, nor irritation of the eye.

A drawback of analogues selective for the EP₂ prostanoid receptor, however, may be that the EP₂ receptor mediates vasodilation (increased blood flow), and thus it can be anticipated that a hyperemic side-effect may appear in the conjunctiva and superficial tissues of the eye. However, this superficial hyperemic side effect of selective EP₂ receptor agonists can be counteracted by the concomitant use of a vasoconstrictive agent, primarily an alpha-adrenergic receptor agonist; either of the alpha-1 or alpha-2 type. Typical alpha-1 adrenergic agonists comprise e.g. phenylephrine and oxymetazoline, and alpha-2 adrenergic agonists e.g. brimonidine, apraclonidine and clonidine. Many of these agents are used in ophthalmogic practice, and an advantage of e.g. brimonidine and apraclonidine is that these substances also reduce the intraocular pressure. Accordingly, the hyperemic side-effect of EP₂ prostanoid receptor agonists may be blunted completely, or at least partly, by using an adrenergic agent that causes vasoconstriction in the superficial ocular tissues.

Useful selective EP₂ prostanoid receptor agonists that have been shown to reduce IOP in animals e.g. in monkeys comprise 19R-hydroxy-PGE₂, butaprost, and AY 23626 (11-deoxy-13,14-dihydro-PGE₁ (U.S. Pat. No. 5,698,598), or prodrugs, e.g. esters and amides of these prostaglandin analogues. Obviously several other prostaglandin analogues of the E-type with selectivity for the EP₂ receptor may be identified by screening and these analogues would have similar beneficial effect in that they should not cause increased pigmentation of the iris.

Accordingly, the present invention in part utilizes previously disclosed information concerning the IOP reducing capacity of EP₂ prostanoid receptor agonists (U.S. Pat. No. 5,698,598), and information concerning the combination of prostaglandins and brimonidine (U.S. Pat. No. 6,294,563) as well as information concerning the ability of alpha-adrenergic receptor agonists to mediate vasoconstriction in the eye. One innovative aspect is the surprising finding that human iridial melanocytes do not express EP₂ prostanoid receptors and thus are not affected by the selective prostaglandin analogues for this receptor. Accordingly, the side effect of increased iridial pigmentation can be avoided, or to a large extent reduced, when selective analogues for the EP₂ prostanoid receptor are used, which is not possible with any of the currently used prostaglandin analogues in the treatment of glaucoma.

The present invention makes available a method for the treatment of glaucoma or ocular hypertension without increased pigmentation of the iris, or with significantly reduced pigmentation of the iris, wherein a prostaglandin analogue selective for the EP₂ prostanoid receptor is used in combination with an alpha-adrenergic agonist. Said prostaglandin analogue is preferably chosen among 19-hydroxy-PGE₂, 19R-hydroxy-PGE₂, butaprost, and 11-deoxy-13,14-dihydro-PGE₁, more preferably among 19-hydroxy-PGE₂ and 19R-hydroxy-PGE₂, and is most preferably 19R-hydroxy-PGE₂.

According to a preferred embodiment, the prostaglandin analogue is an ester or amide prodrug, preferably an ester prodrug, and most preferably an isopropyl ester. It is presently considered that the most preferred prostaglandin analogue is 19R-hydroxy-PGE₂-isopropyl ester.

According to an embodiment of the invention, an alpha-1 adrenergic agonist is used in combination with the prostaglandin analogue. The alpha-1 adrenergic agonist is preferably one of phenylephrine or metaoxedrine, or a combination thereof.

According to another embodiment of the invention, an alpha-2 adrenergic agonist is used in combination with the prostaglandin analogue. The alpha-adrenergic agonist is preferably one of brimonidine, apraclonidine or clonidine, or a combination thereof.

The above substances are used in therapeutically effective and pharmaceutically acceptable amounts.

The present invention also makes available an ophthalmic composition for the treatment of glaucoma or ocular hypertension without increased pigmentation of the iris, or with significantly reduced pigmentation of the iris, wherein said composition comprises a therapeutically effective and pharmacologically acceptable amount of a prostaglandin analogue and a therapeutically effective and pharmacologically acceptable amount of an alpha-adrenergic agonist. Preferably said prostaglandin analogue is chosen among 19-hydroxy-PGE₂, 19R-hydroxy-PGE₂, butaprost, and 11-deoxy-13,14-dihydro-PGE₁, more preferably the prostaglandin analogue is chosen among 19-hydroxy-PGE₂ and 19R-hydroxy-PGE₂, and most preferably the prostaglandin analogue is 19R-hydroxy-PGE₂.

According to a preferred embodiment, the prostaglandin analogue is an ester or amide prodrug, more preferably an ester prodrug, and most preferably an isopropyl ester.

It is presently considered that the most preferred prostaglandin analog is 19R-hydroxy-PGE₂-isopropyl ester

According to an embodiment of the invention, the ophthalmic composition comprises an alpha-1 adrenergic agonist, preferably phenylephrine or metaoxedrine, or a combination thereof.

According to another embodiment, the ophthalmic composition comprises an alpha-2 adrenergic agonist, preferably one or more of brimonidine, apraclonidine or clonidine, or a combination thereof.

The selective prostaglandin analogue should be applied topically on the eye separately or in the same solution with the alpha-adrenergic agonist. Ideally the medication (both the prostaglandin and the alpha-adrenergic agonist) is administered once daily, or even less frequently, e.g. every second day, although twice daily may be necessary depending on the potency of the prostaglandin analogue. The concentration of the prostaglandin analogue should be in the range of 10-1000 micrograms/ml (0.001-0.1%), whereas that of the alpha-adrenergic agent should be in the range of 0.001-0.5%. Brimonidine should be used at a concentration of 0.02-0.2% and apraclonidine at a concentration of 0.1-1%. Formulations of the active components according to the invention may be prepared using methods and adjuvants well know to a person skilled in the art. The active components are preferably admixed with a carrier or vehicle. The vehicle containing the prostaglandin may include a solubilizer such as polysorbate or liposomes to enhance the chemical stability of the prostaglandin. The vehicle may also contain further additives, commonly used in the art, such as preservatives e.g. benzalkonium chloride, chlorhexidine etc., at concentrations suitable for eye drop formulations. Agents increasing the viscosity of the vehicle may also be included, e.g. polyvinylalcohole. The drug formulation according to the present invention may also be applied to the eye using slow release systems such as soluble, or insoluble drug inserts, ointments and alike. The preparation of such drug formulations and drug delivery systems constitutes routine work for a person skilled in the art.

### EXAMPLES

### Materials and Methods

Only methods and results concerned with the demonstration of the lack of expression of EP₂ receptors in human iridial melanocytes are included in the present description. The methods are disclosed in detail by Wentzel et al., 2003. Briefly, iridial melanocytes were isolated from enucleated human eyes, or iridectomy specimens obtained during trabeculectomy surgery. Of the 11 specimens, 6 were obtained from blue eyes, and 5 from hazel eyes. The melanocytes were isolated and cultured as described in more detail by Hu et al., 1997. The tissue was washed thrice with 2 ml HBSS (without Ca²⁺, Mg²⁺) before being placed in 1 ml 0.25% trypsin at 4° C. for 16 hours and after that in 37° C. for 1 hour. Culture medium was added to stop the activity of trypsin. The suspension was centrifuged 200 g×5 min and resuspended in F-12 medium supplemented with 10% FBS, 10 ng/ml cholera toxin, 0.1 mM IBMX, 50 μg/ml gentamicin and 20 ng/ml bFGF. The cell suspension was transferred to a 4 cm² culture dish and placed in an incubator in humidified 95% air and 5% CO₂ at 37° C. The medium was replaced after 2 days and then twice a week. Geneticin was used, if necessary, to eliminate contaminating cells, e.g. fibroblasts and iridial pigment epithelial cells. When the uveal melanocyte cultures were confluent, the cells were passaged using trypsin-EDTA solution (0.05-0.02%), diluted 1:3 and subcultured. The cells were used at passage 3 or 4.

Total RNA was isolated with RNeasy mini kit (QiaGEN, VWR International, Stockholm, Sweden) according to the manufacturer's instructions. Cell pellets were lysed in 350 μl buffer. Thereafter 350 μl of 70% ethanol was added to the homogenates and the samples were mixed and applied to RNeasy mini spin columns, in 2 ml collection tubes. The columns and tubes were centrifuged for 30 sec at 8000 g, the flow through was discarded and the columns were washed with 700 μl buffer (RW 1) and spun at 8000 g for 30 sec. The columns were then washed with 500 μl buffer (RPE) twice and the flow-through was discarded after each centrifugation. The columns were transferred to new collection tubes and 50 μl RNase-free water was applied to each column twice, and the accumulated flow-through was collected after the centrifugations (total RNA sample).

One microgram of total RNA was used for reverse transcription. First strand cDNA synthesis was performed using first strand beads (Ready To Go, Pharmacia Biotech, Uppsala, Sweden), according to the manufacturer's instructions. DEPC-treated water (25-30 μl) containing 1 μg RNA was heated at 65° C. for 10 min and then chilled on ice for 2 min. The RNA solution was transferred to First-Strand Reaction Mix Beads and the primer chosen was Oligo(dT). After incubation at 37° C. for 60 min, heating to 95° C. for 5 min stopped the reaction. The resulting cDNA was diluted 3-fold with DEPC-treated water. One microliter of the cDNA was amplified in a final volume of 20 μl buffer containing 2.5 mmol/l MgCl₂, 0.2 mmol/l dNTP, 25 U/ml Amp-Taq Gold and 5 μg/ml of the sense and antisense primers (Pharmacia Biotech, Uppsala, Sweden). The PCR samples were initially subjected to 10 min incubation at 94° C. Thereafter each of 15 subsequent cycles comprised 30 sec at 94° C. (denaturation), 45 sec at 55° C. (annealing), 45 sec at 72° C. (elongation). From the 16^{th} cycle and onwards 5 sec per cycle was added to the elongation time. In total the samples were run for 38 cycles. Human β-actin was used as housekeeping gene for reference.

The PCR products were run in a 1% agarose gel, stained with ethidium bromide (5 μg/ml) and photographed under UV light (UVP Inc., San Gabriel, Calif. USA). For each set of primers, a number of PCR amplifications with different cycle numbers were tested. The primers for the EP₂ and FP prostanoid receptor genes were purchased from TAG Copenhagen A/S, Copenhagen, Denmark, and the primers for β-actin from Amersham Biosciences, Uppsala, Sweden. The primer sequences were as follows:

Primers for EP₂ receptor:
Forward: 5′-CTT ACC TGC AGC TGT ACG,
and
Reverse: 5′-GAT GGC AAA GAC CCA AAG G
Primers for FP receptor:
Forward: 5′-TTT GAG AGG GAG ATG ACT TGA,
and
Reverse: 5′-GCA CAA CAA TGA AAC ACC AAG
Primers for β-actin:
Forward: 5′-CGA CTA CCT CAT GAA GAT CC,
and
Reverse: 5′-CGA TCC ACA CTG AGT ACT TG

### Results

Only results concerning the expression of the EP₂ and the FP prostanoid receptors are included here. The expression (transcription) of the two prostanoid receptors in human iridial melanocytes can be seen in Table 1.

TABLE 1
Transcription of EP₂ and FP prostanoid receptor genes in human
iridial melanocytes isolated from eyes of different color

| | | |
|---|---|---|
| Sample | Eye color | Prostanoid receptor transcripts |

| Number | of donor | EP₂ | FP |
|---|---|---|---|
| 1 | Blue | − | + |
| 2 | Blue | | |
| 3 | Blue | − | + |
| 4 | Blue | − | + |
| 5 | Blue | − | + |
| 6 | Blue | − | + |
| 7 | Hazel | − | + |
| 8 | Hazel | − | + |
| 9 | Hazel | − | + |
| 10 | Hazel | − | + |
| 11 | Hazel | − | + |

(“+” indicates the presence, and “−” the absence of transcript)

As evident from Table 1, the EP₂ receptor gene was not transcribed in any of the cell cultures whereas the FP receptor gene, with exception of the melanocytes from one individual, was consistently transcribed. Consequently, it is held that selective EP₂ receptor agonists are useful for the treatment of glaucoma in that they should not cause increased pigmentation of the iris, provided that such agonists can be proven effective as IOP reducing agents which has been shown previously (U.S. Pat. No. 5,698,598).

Although the invention has been described with regard to its preferred embodiments, which constitute the best mode presently known to the inventor, it should be understood that various changes and modifications as would be obvious to one having the ordinary skill in this art may be made without departing from the scope of the invention which is set forth in the claims appended hereto.

### REFERENCES

Goodman et al. (Eds.), (1996) Goodman and Gilman's The Pharmacological Basis of Therapeutics”, McGraw-Hill Professional, 9^{th} Ed.
Hellberg, M. R., Sallee, V. L., McLaughlin, M. et al. (2001). Preclinical efficacy of travoprost, a potent and selective FP prostaglandin receptor agonist. J. Ocular Pharmacol. Therapeut. 17: 421-432.
Hellberg, M. R., Ke, T-L, Haggard, K. et al. (2003). The hydrolysis of the prostaglandin analog prodrug bimatoprost to 17-phenyl-trinor-PGF_{2α} by human and rabbit ocular tissue. J. Ocular Pharmacol. Therapeut. 19:97-103.
Hu, D-N., McCormick, S. A., Orlow, S. J., Rosemblat, S. and Lin, A. Y. (1997). Regulation of melanogenesis by human uveal melanocytes in vitro. Exp. Eye Res. 64: 397-404.
Stjernschantz, J. W. (2001). From PGF_{2α}-isopropyl ester to latanoprost: A review of the development of Xalatan. The Proctor Lecture. Invest. Ophthalmol. Vis. Sci. 42: 1134-1145.
Stjernschantz, J., Albert, D. M., Hu, D-N. et al. (2002). Mechanism and clinical significance of prostaglandin-induced iris pigmentation. Surv. Ophthalmol. 47 (Suppl. 1): S162-S175.
Wentzel, P., Bergh, K., Wallin, Ö., Niemelä, P. and Stjernschantz, J. (2003). Transcription of prostanoid receptor genes and cyclo-oxygenase enzyme genes in cultivated human iridial melanocytes from eyes of different colours. Pigment Cell Res. 16: 43-49.
Woodward, D. F., Krauss, A. H.-P., Chen, J. et al. (2001). The pharmacology of bimatoprost (Lumigan™). Surv. Ophthalmol. 45 (Suppl. 4): S337-S345.
Yamamoto, T., Kitazawa, Y., Azuma, I. and Masuda, K. (1997). Clinical evaluation of UF-021 (Rescula^{R}; isopropyl unoprostone). Surv. Ophthalmol. 41 (Suppl. 2): S99-S103.

1. A method for the treatment of glaucoma or ocular hypertension in an individual without increased pigmentation of the iris, or with significantly reduced pigmentation of the iris, comprising administration to the individual of a prostaglandin analogue selective for the EP₂ prostanoid receptor in combination with an alpha-adrenergic agonist.

2. The method according to claim 1, wherein the prostaglandin analogue comprises 19-hydroxy-PGE₂, 19R-hydroxy-PGE₂, butaprost, or 11-deoxy-13, 14-dihydro-PGE₁.

3. The method according to claim 1, wherein the prostaglandin analogue comprises 19-hydroxy-PGE₂ or 19R-hydroxy-PGE₂.

4. The method according to claim 1, wherein the prostaglandin analogue is 19R-hydroxy-PGE₂.

5. The method according to claim 1, wherein the prostaglandin analogue is an ester or amide prodrug.

6. The method according to claim 1, wherein the prostaglandin analogue is an ester prodrug.

7. The method according to claim 6, wherein the prostaglandin analogue is an isopropyl ester.

8. The method according to claim 6, wherein the prostaglandin analogue is 19R-hydroxy-PGE₂-isopropyl ester.

9. The method according to claim 1, wherein an alpha-1 adrenergic agonist is administered in combination with the prostaglandin analogue.

10. The method according to claim 9, wherein phenylephrine or metaoxedrine is administered in combination with the prostaglandin analogue.

11. The method according to claim 1, wherein an alpha-2 adrenergic agonist is administered in combination with the prostaglandin analogue.

12. The method according to claim 11, wherein brimonidine, apraclonidine or clonidine, or two or more thereof, are administered in combination with the prostaglandin analogue.

13. An ophthalmic composition for the treatment of glaucoma or ocular hypertension without increased pigmentation of the iris, or with significantly reduced pigmentation of the iris, wherein said composition comprises a therapeutically effective and pharmacologically acceptable amount of a prostaglandin analogue and a therapeutically effective and pharmacologically acceptable amount of an alpha-adrenergic agonist.

14. The ophthalmic composition according to claim 13, wherein the prostaglandin analogue comprises 19-hydroxy-PGE₂, 19R-hydroxy-PGE₂, butaprost, or 11-deoxy-13, 14-dihydro-PGE₁.

15. The ophthalmic composition according to claim 13, wherein the prostaglandin analogue comprises 19-hydroxy-PGE₂ or 19R-hydroxy-PGE₂.

16. The ophthalmic composition according to claim 13, wherein the prostaglandin analogue is 19R-hydroxy-PGE₂.

17. The ophthalmic composition according to claim 13, wherein the prostaglandin analogue is an ester or amide prodrug.

18. The ophthalmic composition according to claim 13, wherein the prostaglandin analogue is an ester prodrug.

19. The ophthalmic composition according to claim 19, wherein the prostaglandin analogue is an isopropyl ester.

20. The ophthalmic composition according to claim 19, wherein the prostaglandin analogue is 19R-hydroxy-PGE₂-isopropyl ester.

21. The ophthalmic composition according to claim 13, comprising an alpha-1 adrenergic agonist in combination with the prostaglandin analogue.

22. The ophthalmic composition according to claim 21, comprising phenylephrine or metaoxedrine in combination with the prostaglandin analogue.

23. The ophthalmic composition according to claim 13, comprising an alpha-2 adrenergic agonist in combination with the prostaglandin analogue.

24. The ophthalmic composition according to claim 23, comprising brimonidine, apraclonidine or clonidine, or two or more thereof, in combination with the prostaglandin analogue.


SEQUENCE LISTING (6 sequences)

SEQ ID NO: 1 - DNA, 18 bp, Artificial Sequence
  Forward primer for EP2 Receptor
cttacctgcagctgtacg

SEQ ID NO: 2 - DNA, 19 bp, Artificial Sequence
  Reverse Primer for EP2 Receptor
gatggcaaagacccaaaga

SEQ ID NO: 3 - DNA, 21 bp, Artificial Sequence
  Forward Primer for FP Receptor
tttgagagggagatgacttga

SEQ ID NO: 4 - DNA, 21 bp, Artificial Sequence
  Reverse Primer for FP Receptor
gcacaacaatgaaacaccaag

SEQ ID NO: 5 - DNA, 20 bp, Artificial Sequence
  Forward Primer for B-actin
cgactacctcatgaagatcc

SEQ ID NO: 6 - DNA, 20 bp, Artificial Sequence
  Reverse Primer for B-actin
cgatccacactgagtacttg